(19) United States
(12) Patent Application Publication
(10) Pub. No.: US 2017/0079676 A1
(43) Pub. Date: Mar. 23, 2017

(54) HAND HELD DERMAPLANING DEVICE AND METHOD FOR USING SAME

(71) Applicants: Alyssa Lindsey Ceto, West Hollywood, CA (US); Heather Maureen Bradley, Los Angeles, CA (US)

(72) Inventors: Alyssa Lindsey Ceto, West Hollywood, CA (US); Heather Maureen Bradley, Los Angeles, CA (US)

(21) Appl. No.: 15/274,869

(22) Filed: Sep. 23, 2016

Related U.S. Application Data
(60) Provisional application No. 62/222,672, filed on Sep. 23, 2015.
(60) Provisional application No. 62/236,100, filed on Oct. 1, 2015.

(51) Int. Cl. A61B 17/3205

(52) U.S. Cl. CPC A61B 17/3205; A61B 2017/320008

(57) ABSTRACT

In general, the embodiments herein provide an electro/mechanical hand held consumer-level professional-worthy dermaplaning device with replaceable blade heads that scrape, exfoliate, and depilate for more youthful looking skin. The device safely disrupts/abrades the surface of the facial, neck, and décolleté skin and encourages or allows the body's own natural healing process. The device is an electro-gliding tool that sloughs off dead skin and temporally removes vellus hair. The device maximizes product penetration and facilitates shedding of dead skin cells, resulting in more hydrated, moisturized, stronger/responsive, plumper, smoother, younger-looking skin. The device is designed to be used weekly by the consumer, with long term continual use recommended for optimal results.

### CROSS REFERENCE TO RELATED APPLICATION

This application claims priority to U.S. Provisional Patent Application 62/222,672 filed on Sep. 23, 2015, and U.S. Provisional Patent Application 62/236,100 filed on Oct. 1, 2015, the entirety of which are incorporated herein by reference.

### BACKGROUND

Embodiments of the subject matter disclosed herein relate to a hand held dermaplaning device and a method for using such device.

Dermaplaning, also known as Epidermal leveling, is a simple, effective procedure performed by a licensed medical esthetician. It is a gentle form of manual exfoliation of the skin by means of a blade holder attached to a surgical blade or scalpel, that resurfaces sun damaged, dull, uneven skin and removes vellus hair. Through controlled steady maneuvers, the top epidermis layer of dead skin cells, impurities, vellus hair, and skin debris are removed.

This form of gentle trauma sends a message to the brain to ignite, begin, and/or encourage the body's own natural healing process. When the body is in repair mode, it helps the skin to produce a higher level of collagen, micro fiber blast, and other naturally occurring bodily proteins to create stronger, healthier skin. Therefore, dermaplaning is used largely as a beauty treatment among all ages of both males and females. Dermaplaning causes the skin to repair, plump, hydrate and strengthen by means of regular routine removal, therefore creating a wrinkle softening or age defying result.

Other benefits of dermaplaning may include, but are not limited to: reduced pore size; reduced hyper-pigmentation; reduced puffiness; removal of debris, dirt, and/or oil; reduced inflammation; diminished appearance of scars; reduce effects of sun damage; stimulate circulation; enhance penetration of skin care products; and unblock blackheads.

Dermaplaning is typically performed by a licensed professional in a medical office, spa, salon, or other type of location that offers professional dermatology or cosmetology services. Such professionals perform dermaplaning using devices and methods that are not suitable for untrained users or consumers. It may be desirable to have a consumer-level professional worthy device that may be effectively and safely used by untrained or ordinary users thereof.

### BRIEF SUMMARY

The following presents a simplified overview of the example embodiments in order to provide a basic understanding of some aspects of the example embodiments. This overview is not an extensive overview of the example embodiments. It is intended to neither identify key or critical elements of the example embodiments nor delineate the scope of the appended claims. Its sole purpose is to present some concepts of the example embodiments in a simplified form as a prelude to the more detailed description that is presented later.

In accordance with the present disclosure, there is provided a hand held dermaplaning device and a method for using such device.

The dermaplaning device according to the present disclosure is an electro/mechanical hand held consumer-level professional-worthy device with replaceable blade heads that scrape, exfoliate, and depilate for more youthful looking skin. The device safely disrupts/abrades the surface of the facial, neck, and décolleté skin and encourages or allows the body's own natural healing process. The device is an electro-gliding tool that sloughs off dead skin and temporally removes vellus hair. The device maximizes product penetration and facilitates shedding of dead skin cells, resulting in more hydrated, moisturized, stronger/responsive, plumper, smoother, younger-looking skin. The device is designed to be used weekly by the consumer, with long term continual use recommended for optimal results.

In one embodiment, the device will have a secure safety lock guard around the blades that will allow safe usage by the consumer and limit the level of blade penetration into the skin to avoid injury. The device will have an oscillating locked feature that has a predetermined start setting and is controlled by the consumer to allow 2-5 safe levels for use. A secure debris cup allows the flow of dead skin cells and hair to be captured in the cup. An internal fan inside the body of the device will create a slight suction. The debris cup can be emptied and cleaned after each use, or optionally disposed.

In one embodiment, the device will be made up of approximately 6-15 components and the body size will be approximately 5-7 inches in length and ½-4 inches in width. The device is electrically powered by any suitable means, including, but not limited to, battery, rechargeable battery, connection to an electrical current outlet by an attached power cord, and the like.

Still other advantages, aspects and features of the subject disclosure will become readily apparent to those skilled in the art from the following description wherein there is shown and described a preferred embodiment of the present disclosure, simply by way of illustration of one of the best modes best suited to carry out the subject disclosure As it will be realized, the present disclosure is capable of other different embodiments and its several details are capable of modifications in various obvious aspects all without departing from the scope herein. Accordingly, the description will be regarded as illustrative in nature and not as restrictive.

### BRIEF DESCRIPTION OF THE DRAWINGS

The accompanying drawings incorporated herein and forming a part of the specification illustrate the example embodiments.

FIG. 1A depicts front and back views of a dermaplaning device according to one embodiment of the present disclosure.

FIG. 1B depicts a side view of a dermaplaning device according to one embodiment of the present disclosure.

FIG. 1C depicts a side view of a dermaplaning device according to one embodiment of the present disclosure.

FIG. 2A and FIG. 2B depict a charging unit for a dermaplaning device according to one embodiment of the present disclosure.

FIGS. 3A-H depict several embodiments of blade configurations for a dermaplaning device according to the present disclosure.

### DETAILED DESCRIPTION

This description provides examples not intended to limit the scope of the appended claims. Reference in the specification to “one embodiment” or “an embodiment” or “an example embodiment” means that a particular feature, structure, or characteristic described is included in at least one embodiment described herein and does not imply that the feature, structure, or characteristic is present in all embodiments described herein.

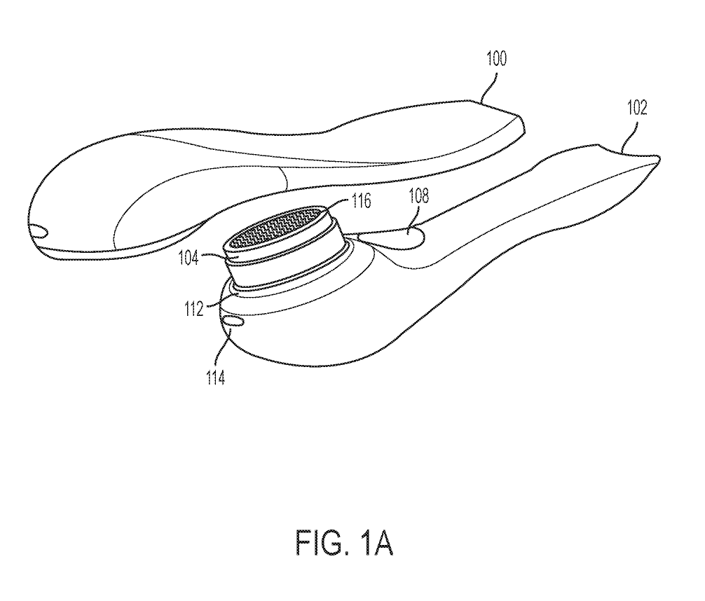

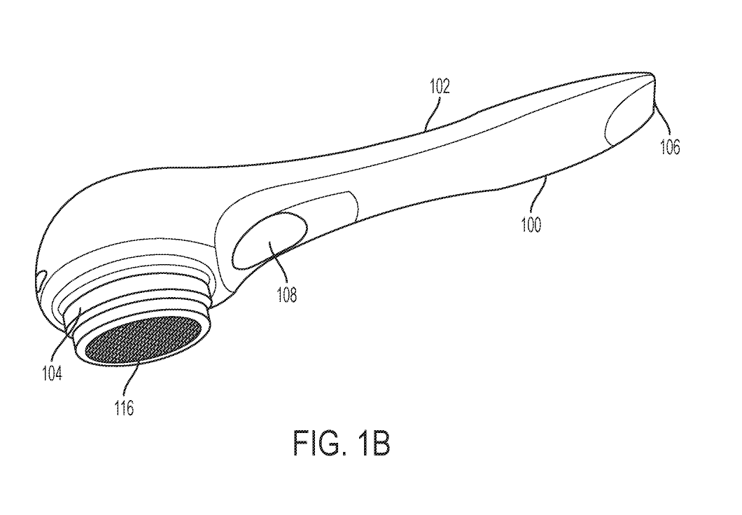

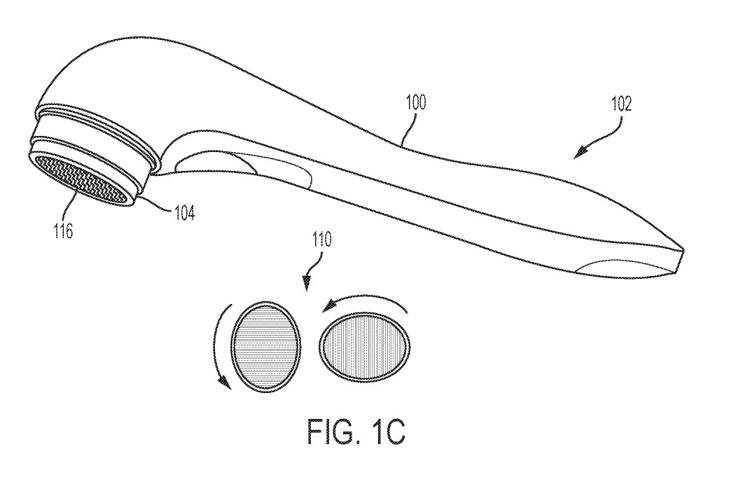

In general, the embodiments herein provide an electro/mechanical hand held consumer-level professional-worthy dermaplaning device with replaceable blade heads that scrape, exfoliate, and depilate for more youthful looking skin. The device safely disrupts/abrades the surface of the facial, neck, and décolleté skin and encourages or allows the body's own natural healing process. The device is an electro-gliding tool that sloughs off dead skin and temporally removes vellus hair. The device maximizes product penetration and facilitates shedding of dead skin cells, resulting in more hydrated, moisturized, stronger/responsive, plumper, smoother, younger-looking skin. The device is designed to be used weekly by the consumer, with long term continual use recommended for optimal results. Various embodiments of the device are contemplated, as discussed below and illustrated in the FIGS. 1-3.

As shown in FIGS. 1A-C, the device 100 includes a body 102 having an blade housing 104 attached thereto, wherein the blade housing includes at least one blade therein. The body is sized and configured to be grasped and held in a human hand, like an electric shaver. The body includes a driving means (not shown) for moving/oscillating/rotating the at least one blade for dermaplaning. The blades may be moved/oscillated/rotated at any speed suitable to achieve the result desired by the user. The driving means are any suitable means known in the art for moving the blades. In one embodiment, the body includes an electric motor (not shown) connected to the driving means and operable to drive the driving means. Preferably the device includes a fan (not shown) to keep the device cool and help create a tornado effect that encourages air suction up the body of the device through the blade housing and pulls the debris into an attached debris cup.

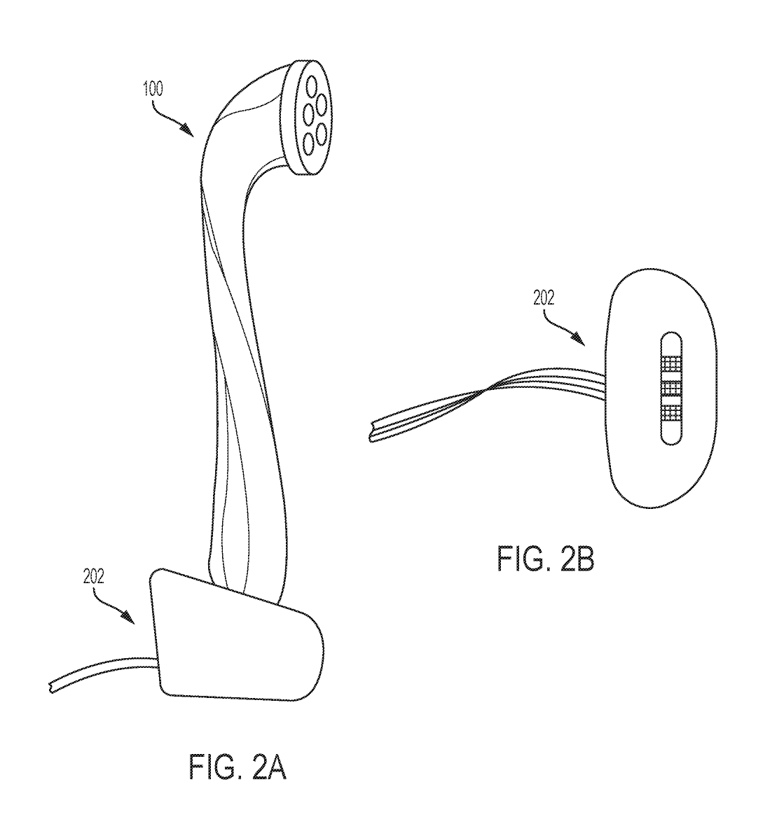

The device is electrically powered by any suitable means, including, but not limited to, battery, rechargeable battery, connection to an electrical current outlet by an attached, preferably retractable, power cord, and the like. In one embodiment, the device is suitably operable with 110 Voltage electrical power systems, 220 Voltage electrical power systems, or both. In one embodiment, wherein the device is powered by a rechargeable battery, the rechargeable battery may be recharged by any suitable means, including, but not limited to, removing the battery and placing it in a charging mechanism or pod, placing the device with the battery intact into a charging mechanism or pod, connecting the device/battery to an electrical current outlet by an attached, preferably, retractable, power cord, or any other means known in the art. The device may also include an extended life rechargeable battery, which may be recharged in the charging mechanism or pod as shown in FIGS. 2A-B as 202. In one embodiment, the device includes a power input 106 to connect to such power means.

An on/off switch 108 is located on the device for switching the motor on and off, thereby starting and stopping the driving means. The on/off switch preferably includes a locking mechanism for locking the switch in the on or off position, as desired by the user. In a preferred embodiment, the on/off switch includes a sliding or push button speed control lever which is operable to control the speed of the driving means, and hence, movement of the blades. In a preferred embodiment, the sliding speed control lever includes a locking mechanism for locking the lever at a specified speed desired by the user.

The device body is preferably fitted to hold in the hand, and is comprised of any suitable material. Preferably, the device is comprised of a soft, tough metal, rubberized, or plastic material, or a combination thereof. Such material preferably allows for stop-stick grip for secure hand, palm, thumb, and finger control of the device. The device body may also be comprised of recycled materials. In one embodiment, the device will be made up of approximately 6-15 components and the body size will be approximately 5-7 inches in length and ½-4 inches in width.

The device includes a blade housing 104 attached to the top of the body. The blade housing may be constructed such that is used only once and then disposed, or constructed for multiple uses. In a preferred embodiment, the blade housing may be moved into multiple positions for a controlled, comfortable, effective easy to maneuver/pass onto the face, down the neck and over the décolleté, allowing the blades attached thereto to closely hover/grip/glide onto or over the user's skin/face as referenced at 110. Preferably, the device includes a locking mechanism for locking the blade housing into a specified position desired by the user.

The blade housing preferably includes a debris cup 112 attached thereto for collecting dead skin, hair, and other debris resulting from the use of the device. The debris cup is comprised of plastic or other suitable material. The debris cup may be constructed such that is used only once and then disposed, or constructed for multiple uses. In one embodiment, the devices includes a mechanism, such as button as shown at 114, for releasing the debris cup from the device.

The blade housing 104 includes at least one blade, preferably, a number of blades, attached therein. The blade housing and the blades may be constructed as a single unit, such as cartridge, or the like, with the blades fixedly contained within the housing. In such an embodiment, the blade housing with the blades contained therein would be attached to the device body by any suitable means. In another embodiment, the blade housing and the blades are separate components, with the blades attached within the blade housing by any suitable means. In one embodiment, the blade housing is attached to the device body, either permanently, semi-permanently, or with each use, and the blades are attached therein thereafter. In another embodiment, the blades may be attached within the blade housing prior to attachment of the blade housing onto the device body.

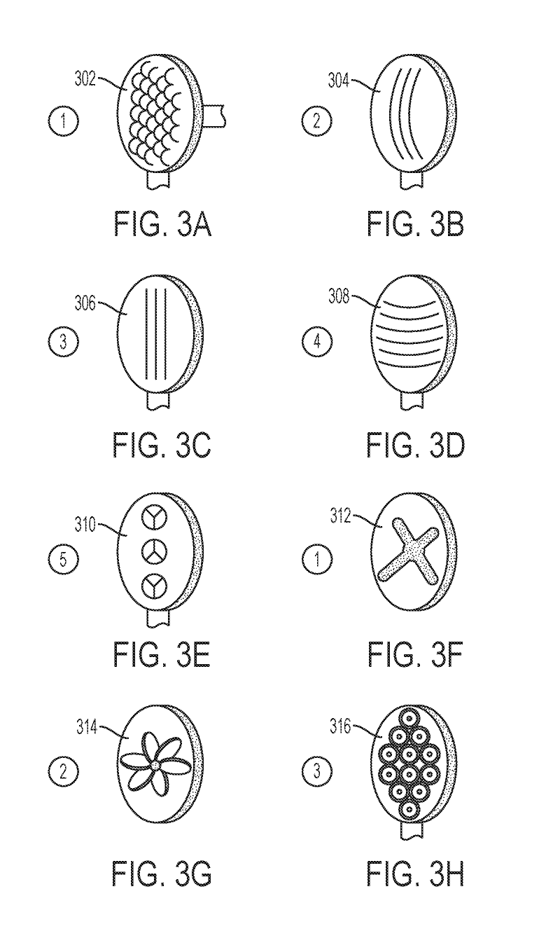

The blade housing includes a secure safety lock guard 116 around the blades that will allow safe usage by the consumer and limit the level of blade penetration into the skin to avoid injury. Various embodiments of the number of blades attached to the blade housing and the configuration thereof are contemplated as discussed herein and as illustrated in FIGS. 3A-H. The blades may be attached/detached to the blade housing by any means known in the art, such as a snap on/snap off mechanism, slotted grooves, projection/aperture, and the like. The blades and/or the blade housing are designed to be detached after each use or multiple uses, and discarded. The blades are attached such that they are able to move/oscillate/rotate as desired from the operation of the drive means, resulting in a removal, sloughing, and scraping of hair and dead skin off the epidermis layer. Preferably, the blades move/oscillate/rotate in one direction.

The blades may be of any size, shape, or material as is known in the art to effectively dermaplane a user's skin. The shape of the blades include, but are not limited to, flat, ½ moon shaped, round, oval, oblong, rectangular, circular, and straight. The types of blades contemplated include, but are not limited to: carbon steel blades, circular carbon steel blades, disc blades, double edge blades, fine gauge blades, fine slant blades, oval feather slanted blades, oval shaped blades, oval shaped carbon steel blades, pinwheel feather slanted blades, rectangular feather slanted blades, round feather slanted blades, serrated blades, straight blades, and straight feather slanted blades.

FIGS. 3A-H illustrate various configurations of blades contemplated by the present disclosure. FIG. 3A illustrates a half moon configuration 302. FIG. 3B illustrates a long moon shape tightly overlapped, configuration 304 which hugs the base of the debris cup 112. FIG. 3C illustrates a straight blade configuration 306 having 3 to 5 blades. FIG. 3D illustrates an overlapping blade configuration 308 having 6 to 8 overlapping blades. FIG. 3E illustrates a miniature windmill rotating blade configuration 310 having which hug closely to the base of the blade housing 104. While the embodiment shows 3 blades, such configuration may have any number of blades. FIG. 3F illustrates a windmill blade configuration 312. FIG. 3G illustrates a clover petal blade configuration 314. FIG. 3H illustrates a honeycomb circular blade configuration 316.

In another embodiment, the device has a 3 dimensional raised blade housing 104 or mechanism. The blade housing is any desired shape, including, but not limited to, oval, half mooned shape, round, flat, rectangular, oblong, circular, or straight. The blade housing 104 has retractable blades that sit within or in the center of the blade housing. The blades are retracted in any suitable manner, such as by operation of the driving means or the like. The blade housing has the ability to protrude, raise, or project in a 3 dimensional way. When retracted, the blades slide in evenly to create a solid smooth, cold steel/metal surface that activates a cooling element for effective gliding of product, pressure to be applied for smoothing soothing effects on the skin, or both. The cooling element is any suitable mechanism known in the art, preferably, a smooth steel plate or face that provides cooling to the skin by a cooling means associated therewith. This helps to treat inflammation, cools, soothes, helps reduce the appearance of pores, and allows a skin tightening effect.

In a preferred embodiment, the device includes a face cape or protective head band that keeps the device away from the user's hairline. The device may also include a travel bag.

The device may be used on dry skin or wet skin as desired by the user.

By regular weekly long term use, the consumer may receive the following benefits:

Exfoliation

Debridement

Scraping

Shaving

Superior cleansing of the skin

Improved appearance of skin texture and tone

Diminished appearance of fine lines and wrinkles

Smooths the appearance of skin texture

Temporary removal of vellus hair—will not grow back thicker

Encourages dead skin cell removal

Encourages the generation of new healthy skin cell turnover

Better product absorption into the skin that leads to better product efficacy

Safe healthy trauma to the skin by exfoliation at a safe level without harming healthy skin tissue.

Having thus described certain embodiments for practicing aspects of the present disclosure, it is to be appreciated that various alterations, modifications, and improvements will readily occur to those skilled in the art. Such alterations, modifications, and improvements are intended to be part of this disclosure, and are intended to be within the spirit and scope of this disclosure.

1. A hand-held dermaplaning device comprising:
a body having an upper end and a bottom end, wherein the body is shaped to fit at least partially within the hand of an associated user;
a blade housing intended to be moved in contact with the skin and attached to the upper end of the body, wherein the blade housing includes an interior and an exterior;
at least one blade attached to the interior of the blade housing, wherein the at least one blade is attached in such a manner to allow movement of the at least one blade within the blade housing during operation of the device; wherein the at least one blade has at a free end a single cutting edge suitable for dermaplaning the skin;
a security safety lock guard positioned at least partly on the cutting edge of the at least one blade to limit the level of blade penetration into the skin;
a driving source coupled to the blade housing and configured to drive the movement of the at least one blade; and
an electrical power source coupled to the driving source and configured to provide electric current to the driving source for operation thereof.

2. The hand-held dermaplaning device of claim 1, further comprising a switch selectively operable between an ON status for allowing the electric current flow from the electric power source to the driving source and an OFF status for interrupting the electric currently flow to the driving source.

3. The hand-held dermaplaning device of claim 2, wherein the switch further comprises a locking device configured to lock the switch in the ON status or the OFF status.

4. The hand-held dermaplaning device of claim 2, wherein the switch further comprises a speed control leve4 operable to control the speed of the driving source.

5. The hand-held dermaplaning device of claim 4, wherein the speed control lever includes a speed control lever lock operable to lock the lever at a specified speed.

6. The hand-held dermaplaning device of claim 1, wherein the electrical power source is selected from the group consisting of a battery, a rechargeable battery, a connection to an electrical current outlet, and combinations thereof.

7. The hand-held dermaplaning device of claim 1, wherein the body size ranges from approximately 5 to 7 inches in length and from approximately ½ to 4 inches in width.

8. The hand-held dermaplaning device of claim 1, wherein the blade housing is attached to the body in such a manner that the blade housing is able to move in a plurality of positions with respect to the body.

9. The hand-held dermaplaning device of claim 8, further comprising a blade housing lock operable to lock the blade housing in a specified position with respect to the body.

10. The hand-held dermaplaning device of claim 1, wherein the blade housing has a shape with is substantially oval, a half moon shaped, round, flat, rectangular, oblong, circular, or straight.

11. The hand-held dermaplaning device of claim 1, wherein the at least one blade is fixedly attached to the interior of the blade housing.

12. The hand-held dermaplaning device of claim 1, wherein the at least one blade is detachably attached to the interior of the blade housing for removal thereof.

13. The hand-held dermaplaning device of claim 1, wherein the at driving source is operable to move the at least one blade in only one direction.

14. The hand-held dermaplaning device of claim 1, wherein the at least one blade has a shape that is substantially flat, half moon shaped, round, oval, oblong, rectangular, circular, or straight.

15. The hand-held dermaplaning device of claim 1, wherein the at least one blade is selected from the group consisting of: carbon steel blades, circular carbon steel blades, disc blades, double edge blades, fine gauge blades, fine slant blades, oval feather slanted blades, oval shaped blades, oval shaped carbon steel blades, pinwheel feather slanted blades, rectangular feather slanted blades, round feather slanted blades, serrated blades, straight blades, straight feather slanted blades, and combinations thereof.

16. The hand-held dermaplaning device of claim 1, further comprising a blade retraction source operable to retract the at least one blade attached within the interior of the blade housing during operation of the device.

17. The hand-held dermaplaning device of claim 1, wherein the blade housing further comprises a debris cup attached thereto operable to collect debris generated by the operation of the device.

18. The hand-held dermaplaning device of claim 1, further comprising a fan attached to the body operable to create a tornado effect that generates air suction up the body of the device through the blade housing.
